(19) United States
(12) Patent Application Publication
(10) Pub. No.: US 2022/0238210 A1
(43) Pub. Date: Jul. 28, 2022

(54) METHOD AND SYSTEM FOR REDUCING HOSPITAL SUPPLY COSTS USING MIXED INTEGER LINEAR PROGRAMMING

(71) Applicant: Sam Houston State University, Huntsville, TX (US)

(72) Inventors: Liu Yang, Conroe, TX (US); Mitchell A. Millstein, Chesterfield, MO (US)

(21) Appl. No.: 17/585,371

(22) Filed: Jan. 26, 2022

Related U.S. Application Data
(60) Provisional application No. 63/142,455, filed on Jan. 27, 2021.

(51) Int. Cl. G16H 40/20; G06Q 30/02; G06Q 10/08

(52) U.S. Cl. CPC G16H 40/20; G06Q 10/0875; G06Q 30/0206

(57) ABSTRACT

System and methods are provided for reducing medial supply costs for healthcare providers by effectively leveraging tier pricing programs brought forth by suppliers. Tier contracts offered by healthcare clinical products suppliers are one of the most complex pricing schemes with many distinctive challenges. A hospital supply spend optimization system is disclosed that minimizes medical supply costs for healthcare providers, suggests optimal spends on individual suppliers by determining product and quantity to be purchased from each supplier, evaluates tiers qualified and assigns optimal tier prices to member hospitals, and recommends product substitution structure for cost minimization. The system may also be used to quantify the impact of physician preferences on hospital supply costs.

### BACKGROUND

### 1. Field of the Invention

The disclosed embodiments generally relate to systems and methods used to reduce medial supply costs for healthcare providers by effectively leveraging tier pricing programs brought forth by suppliers.

### 2. Description of the Relevant Art

Hospital spending is the primary driver of the increasing healthcare spending in the U.S., costing over one trillion dollars. The high healthcare cost erodes disposable income and the wealth of many American families. Hospitals, in the meantime, struggle to stay financially viable. Many large healthcare systems have observed double-digit growth in revenue in the past few years, but revenue growth is outpaced by a much faster increasing operating expense, which eventually leads to declined operating income. A close examination of hospital cost structure reveals that spending on tangible medical supplies alone may account for 30% to 40% of a hospital's expenses, the second largest cost category after labor, and it is growing faster than labor costs. As such, it is critical for healthcare organizations and group purchasing organizations (GPOs) to have a method and system that allows them to make optimal purchasing decisions to minimize the procurement cost of medical supplies.

Healthcare organizations establish contracts with a set group of healthcare clinical products suppliers, either directly or through GPOs. These suppliers are called contract suppliers or contract vendors. Contract vendors offer healthcare organizations complicated contracts that have multiple pricing tier options, known as tier pricing. Tier pricing is a form of quantity discount. Suppliers offer quantity discounts to reduce operating costs, induce larger purchases, and/or gain market share. It is well studied in the supply chain and marketing literature that quantity discount in general also improves the profits of buyers. This benefit, however, has not been realized for healthcare providers.

Tier contracts offered by healthcare clinical products suppliers are one of the most complex pricing schemes with many distinctive challenges. First, a tier requirement is often built upon multidimensions, involving consumption level, market share, the number of suppliers, and facility and IDN level of measures. These dimensions are intertwined, creating a few to more than a dozen of tier prices in each contract offered by suppliers. In addition, many suppliers segment their product lines and negotiate contracts on individual product categories; for example, a supplier may have a three-tier contract in total joint implants but a completely different seven-tier contract in electrophysiology products. A large healthcare organization may have dozens of categories; within a category are a variety of pricing schedules offered by individual manufacturers. Moreover, a category-based contract may cover dozens to over a hundred of items with varying discount rates for the same tier. This is in contrast to other industries where suppliers typically apply the same discount rate across all products. Furthermore, within a contract, multiple facility, IDN, and even GPO level tiers coexist along with various market share requirements based on different number of suppliers. An IDN-level tier is not necessarily better than a facility-level tier, barring the same market share requirements. Facilities in an IDN may opt for different facility-level tiers, but the IDN as a whole cannot have both facility and IDN tiers from the same contract. Therefore, a facility may have to abnegate a favorable facility-level tier in order for the IDN to achieve a lower total cost. The GPO-level tiers follow the same rule, except that it is very difficult to meet the onerous market share requirements. To realize the significant cost saving opportunities provided by tier pricing, healthcare organizations need a new and innovative method and system to evaluate all tier options in a holistic manner, optimize product and supplier selection decisions, and eventually minimize the procurement cost of medical supplies.

Another challenge that hospitals face is physician preference items (PPIs). Purchasing decisions in most industries are driven by product quality, cost, and supplier's service commitment. Hospital procurement is different and strongly influenced by physician preferences. Many of the most expensive supplies in surgery procedures, such as cardiac, orthopedic, and spine, are items for which physicians have strong preferences. Physicians generally possess limited knowledge about the cost of medical supplies and often underestimate the cost of high-cost items. Physician preference is widely criticized for inflating supply costs, which has motivated considerable amount of research addressing hospital-physician conflicts and organizational alignments. A core question that has yet to be answered is to what extent exactly physician preferences impact a hospital's bottom line, which PPIs shall be standardized, and to what vendors' products the PPIs be standardized. Studies show that physicians are more willing to work with hospitals on product standardization when they are informed of the cost implication of alternative products.

### BRIEF DESCRIPTION OF THE DRAWINGS

Embodiments disclosed herein are not limited to any specific devices. The drawings described herein are for illustration purposes only and are not intended to limit the scope of the embodiments.

FIG. 1 is a schematic representation of tier contracts between an IDN and its contract vendors regarding a clinical product category.

FIG. 2 depicts a schematic diagram of the process architecture for optimizing procurement decisions, quantifying the impact of physician preferences, and minimizing hospital supply spend, according to some embodiments of the method disclosed.

FIG. 3 shows an example change in spends on a healthcare clinical product category after applying system and methods disclosed to a regional IDN.

FIG. 4 illustrates an example computer-implemented environment that optimizes procurement decisions for healthcare providers.

FIG. 5 is a flowchart illustrating an example process for validating tier prices that hospitals are paying.

FIG. 6 is a flowchart depicting an example process for evaluating the impact of physician preferences.

FIG. 7 is a flow diagram illustrating a method for reducing hospital supply costs, according to some embodiments.

FIG. 8 is a block diagram of one embodiment of a computer system.

Although the embodiments disclosed herein are susceptible to various modifications and alternative forms, specific embodiments are shown by way of example in the drawings and are described herein in detail. It should be understood, however, that drawings and detailed description thereto are not intended to limit the scope of the claims to the particular forms disclosed. On the contrary, this application is intended to cover all modifications, equivalents and alternatives falling within the spirit and scope of the disclosure of the present application as defined by the appended claims.

This disclosure includes references to “one embodiment,” “a particular embodiment,” “some embodiments,” “various embodiments,” or “an embodiment.” The appearances of the phrases “in one embodiment,” “in a particular embodiment,” “in some embodiments,” “in various embodiments,” or “in an embodiment” do not necessarily refer to the same embodiment. Particular features, structures, or characteristics may be combined in any suitable manner consistent with this disclosure.

Reciting in the appended claims that an element is “configured to” perform one or more tasks is expressly intended not to invoke 35 U.S.C. § 112(f) for that claim element. Accordingly, none of the claims in this application as filed are intended to be interpreted as having means-plus-function elements. Should Applicant wish to invoke Section 112(f) during prosecution, it will recite claim elements using the “means for” [performing a function] construct.

As used herein, the term “based on” is used to describe one or more factors that affect a determination. This term does not foreclose the possibility that additional factors may affect the determination. That is, a determination may be solely based on specified factors or based on the specified factors as well as other, unspecified factors. Consider the phrase “determine A based on B.” This phrase specifies that B is a factor that is used to determine A or that affects the determination of A. This phrase does not foreclose that the determination of A may also be based on some other factor, such as C. This phrase is also intended to cover an embodiment in which A is determined based solely on B. As used herein, the phrase “based on” is synonymous with the phrase “based at least in part on.”

As used herein, the phrase “in response to” describes one or more factors that trigger an effect. This phrase does not foreclose the possibility that additional factors may affect or otherwise trigger the effect. That is, an effect may be solely in response to those factors, or may be in response to the specified factors as well as other, unspecified factors.

As used herein, the terms “first,” “second,” etc. are used as labels for nouns that they precede, and do not imply any type of ordering (e.g., spatial, temporal, logical, etc.), unless stated otherwise. As used herein, the term “or” is used as an inclusive or and not as an exclusive or. For example, the phrase “at least one of x, y, or z” means any one of x, y, and z, as well as any combination thereof (e.g., x and y, but not z). In some situations, the context of use of the term “or” may show that it is being used in an exclusive sense, e.g., where “select one of x, y, or z” means that only one of x, y, and z are selected in that example.

In the following description, numerous specific details are set forth to provide a thorough understanding of the disclosed embodiments. One having ordinary skill in the art, however, should recognize that aspects of disclosed embodiments might be practiced without these specific details. In some instances, well-known, structures, computer program instructions, and techniques have not been shown in detail to avoid obscuring the disclosed embodiments.

### DETAILED DESCRIPTION OF THE PREFERRED EMBODIMENTS

Throughout the document, the terms “medical supplies”, “hospital supplies”, “healthcare clinical products”, and “clinical products” are used interchangeably. The terms “healthcare system” and “healthcare organization” are used to describe a healthcare integrated delivery network (IDN) or integrated delivery system (IDS). The term “hospitals” and “facilities” are used interchangeably, referring individual healthcare facilities within an IDN unless the content clearly states otherwise. The term “healthcare providers” is a general term that refer to IDNs, hospitals under an IDN (e.g., “member hospitals”), and other types of healthcare facilities. The terms “suppliers”, “vendors”, and manufacturers” are used interchangeably throughout this disclosure.

It is to be understood the disclosed embodiments are not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Embodiments disclosed herein present a system and method to help healthcare organizations quantify the financial impact of PPIs, standardize healthcare clinical products, and most importantly, reduce the costs of medical supplies. Significant cost saving opportunities are offered not only to large healthcare systems with dozens or even hundreds of facilities, but also to small healthcare providers that have one or two facilities. It is particularly valuable to the healthcare organizations where purchasing decisions are centralized, either within the organization or through a GPO, and are to be optimized at the organization level instead of for individual facilities.

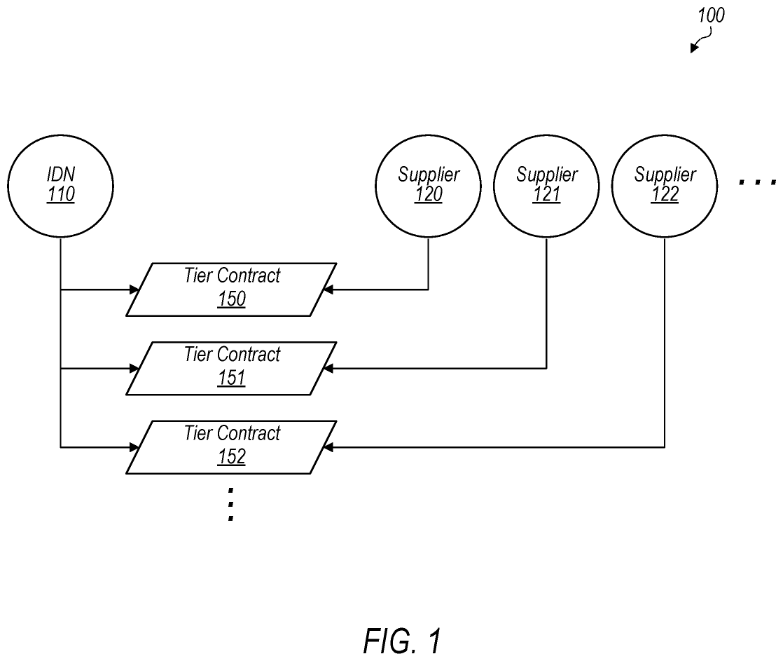

FIG. 1 is a schematic view of system 100 where tier contracts 150, 151, and 152 are negotiated and agreed between an IDN 110 and individual suppliers 120, 121, and 122 for a healthcare product category. Tier contracts are prepared by suppliers. Each contract has a unique tier pricing structure that specifies tier conditions and tiered product prices offered to hospitals if tier conditions are met.

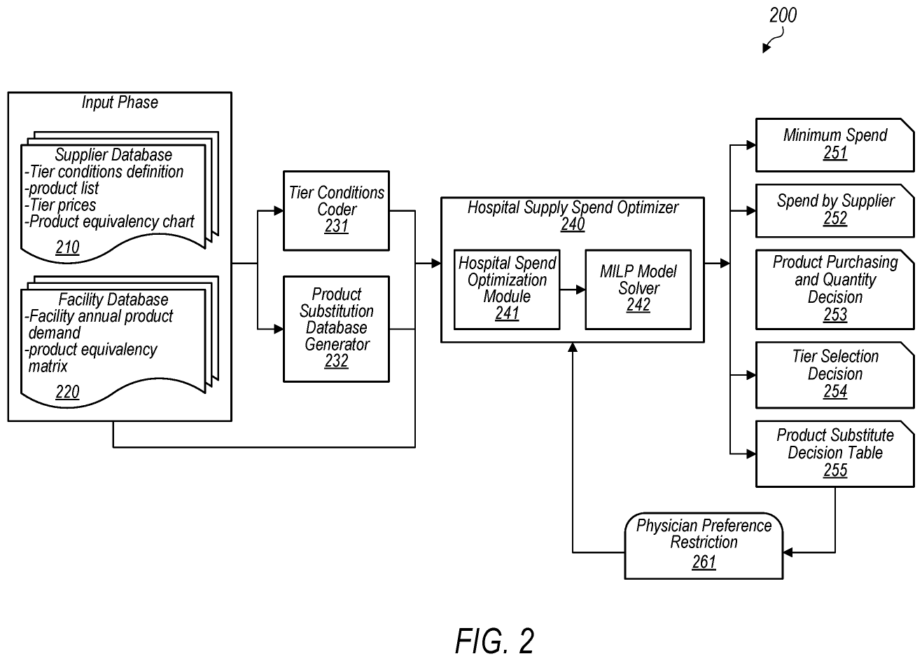

FIG. 2 illustrates an exemplary embodiment of the hospital supply spend optimization system 200. The details of a tier contract including tier conditions, product list, and tier prices, are part of the supplier database 210 hosted at a healthcare organization. Each supplier is asked to provide a product equivalency chart that shows which products it offers are alternatives to products offered by its competitors. Facility database 220 stores product demand information of each hospital and product equivalency matrix known to hospitals. A tier conditions coder 231 retrieves tier condition requirements from the supplier database 210, converts tier definitions to multi-dimensional vectors, and provides this information to the hospital supply spend optimizer 240. For example, a tier condition in one of the tier contracts may state that two or fewer of the contract vendors must have a minimum of 80% of the combined unit market share in a healthcare clinical product category at a hospital level during the contract period, in order for the hospital to qualify for this tier. Another tier condition in the same contract may state that this supplier must have a unit market share of 80% or higher among all the vendors that offer products to the IDN. And there is another tier stating that three or fewer of the contract vendors must have a combined unit market share of 90% or higher at the IDN level, and this supplier itself must have a minimum of 70% of the market share in the IDN.

A tier conditions coder 231 transforms the above tier requirements into six-dimensional vectors and each vector comprises supplier name, tier name, base of the market share requirement (i.e., facility level or IDN level), minimum unit market share requirement of this supplier, maximum number of vendors in the combined market share requirement, and minimum combined unit market share requirement. One of the major challenges to capture the cost saving opportunities is the complex structure of tier conditions, which are made up of a number of factors (e.g., the number of suppliers, market share, etc.) and have no standards. The coder 231 standardizes the structure of tier requirements and provide distinct advantages described herein. For instance, in some embodiments, standardization of the structure of tier requirements using multi-dimensional vectors may allow a computer system to assess the tier requirements (e.g., tier conditions) more efficiently in determining and solving for various parameters such as costs of hospital supplies, as described herein. Without the use of multi-dimensional vectors, solving for the various described parameters using a computer system may be time-consuming and costly and involve various manual inputs that can slow down the process and reduce efficiency of the computer system.

A product substitution database generator 232 generates a one-way product substitution database by analyzing the product equivalency charts provided by suppliers (stored in supplier database 210), and the equivalency matrix known to hospitals (stored in facility database 220). Healthcare clinical products, such as coronary stents and artificial knees, have stringent quality requirements, are highly complex, require special handling, and are modified frequently. Many hospitals have established teams consisting of doctors, nurses, and procurement specialists to assess products and their equivalency relationships, but rapid technology and medical innovations make it hard for hospitals to keep track of all the changes and new functionalities of clinical products. The problem is aggravated by manufacturers' unwillingness to share data, meticulously making their products unique, and blocking products from being comparable. As a result, hospitals often have to rely on manufacturers to indicate alternative products that they offer to substitute products provided by competitors. And since no two products are exactly identical and a seemingly trivial variation may compromise the safety and outcome of patients, similar products from different manufacturers generally cannot be assumed interchangeable. This is a unique phenomenon in healthcare due to the criticality nature of clinical products; for example, product A may substitute product B, but a reverse substitution is not allowed. In addition, not all products have equivalents; a supplier may have equivalents for some of the products offered by another supplier but can rarely replace a competitor's entire product portfolio, even only for a single clinical category. This makes the supplier selection especially challenging as any product substitute decisions made towards a supplier must take into account the impact on the cost of the remaining products that have no alternatives.

A plurality of vectors (storing tier requirements) generated by the tier conditions coder 231, the product substitution mapping generated by the product substitution database generator 232, the product lists and the tier prices extracted from the supplier database 210, and the hospitals' product demand data extracted from the facility database 220, are provided to a hospital supply spend optimizer 240, which comprises a hospital spend optimization module 241 and a mixed integer linear programming (MILP) model solver 242. The optimizer 240 calculates the minimum medical supply costs 251 for each hospital and the IDN as a whole, optimal spends on individual suppliers 252, product and quantity to be purchased from each supplier 253, tier prices qualified and assigned to each facility 254, and product substitute decision table 255. The product substitute decision table 255 is reviewed by physicians. If a physician insists on using the current clinical products (i.e., physician preferred items), a physician preferences constraint 261 is input to the optimizer 240.

The core of the hospital spend optimization module 241 is a MILP optimization model that minimizes the total acquisition cost of healthcare clinical products for an IDN as a whole. To describe the MILP model, consider an IDN comprising a set of hospitals, denoted by H. The purchasing decision is centralized at the IDN level. Let S be the set of suppliers. Each supplier s E S offers a discount schedule: K^{s}={1, 2, . . . }, indexed by k. A tier kϵK^{s} is delineated by k(u,v,w,z), where u describes the required minimum market share of vendor s in a clinical product category, v represents the least combined market share, w indicates the maximum number of vendors for the combined market share, and z shows whether the market share is measured at a facility or an IDN level.

Let I be the set of products including the items currently purchased by hospitals and all the products offered by suppliers. The supplier sϵS offers a portfolio of products I^{s}ϵI. An array R records one-way product substitution relationships; a pair (i,j)ϵR indicates that item jϵI^{s}^{₂}(s₂ϵS) is capable of substituting item iϵI^{s}^{₁} (s₁ϵS). The demand of a facility hϵH for an item iϵI is described by dₕᵢ, which can be forecast based on actual consumptions in previous years. The price of an item iϵI^{s} quoted at a tier kϵK^{s} is denoted as pᵢₖ.

To minimize the total costs of medical supplies for an IDN, the hospital spend optimization module 241 minimizes the following equation:

Σ_{hϵH}Σ_{sϵS}Σ_{iϵI}_{^{s}}Σ_{kϵK}_{^{s}}xₕᵢₖpᵢₖ (1)

where

xₕᵢₖ is the optimal quantity to be purchased by facility h at tier price k regarding item i, ∀hϵH, iϵI^{s}, kϵK^{s}, sϵS.

Expression (1) calculates the total costs of medical supplies in an IDN.

The optimization module 241 takes into account a plurality of constraints, including:

Σ_{kϵK}_{^{s}}eₕₛₖ≤1 ∀hϵH,sϵS (2)

Σ_{iϵI}_{^{s}}xₕᵢₖ≤M·eₕₛₖ ∀hϵH,sϵS,kϵK^{s} (3)

Σ_{jϵI}yₕᵢⱼ≥dₕᵢ ∀hϵH,iϵI,(i,j)ϵR (4)

Σ_{kϵK}_{^{s}}xₕᵢⱼ≥Σ_{iϵI}yₕᵢⱼ ∀hϵH,sϵS (5)

where

eₕₛₖ is a binary variable; eₕₛₖ equals 1 if facility h is assigned to tier k of supplier s, and 0 otherwise, ∀hϵH, sϵS, kϵK^{s}.

yₕᵢⱼ is the optimal quantity of item i to be substituted by item j at facility h, ∀hϵH, iϵI^{s}, kϵK^{s′}, s, s′ϵS, s≠s′.

A facility in an IDN may qualify for multiple tiers offered by a supplier, but a facility shall be assigned at most one tier of a given supplier. Constraint (2) achieves this purpose by restricting a facility from having more than one tier selected from a supplier. Constraint (3) guarantees that no quantity is allocated to a tier which a facility does not qualify for or adopt. Constraints (4) and (5) state that all the demand must be satisfied.

Suppliers typically offer two levels of discount schedules: facility schedule and IDN schedule. Consider first the facility-based discount schedule, indicated by k(z)=“facility”. The following constraint ensures that if a tier k is selected, the total quantity purchased by a facility from supplier s must meet the minimum market share requirement placed on that tier. The minimum market share requirement of the supplier at tier k is indicated by k(u).

Σ_{iϵI}_{^{s}}Σ_{kϵK}_{^{s}}xₕᵢₖ−Σ_{sϵS}Σ_{iϵI}_{^{s}}Σ_{kϵK}_{^{s}}xₕᵢₖ·k(u)≥(eₕₛₖ−1)·M

∀hϵH,kϵK^{s},sϵS (6)

If hospitals are restricted from purchasing more volume than the forecast demand dₕᵢ, the minimum market share requirement in constraint (6) can be simplified to a fixed minimum quantity and rewritten as:

Σ_{iϵI}_{^{s}}Σ_{kϵK}_{^{s}}xₕᵢₖ−Σ_{iϵI}dₕᵢ·k(u)≥(eₕₛₖ−1)·M

∀hϵH,kϵK^{s},sϵS (7a)

The minimum combined market share requirement typically involves another one or two suppliers in addition to supplier s. When k(w)=2 (i.e., the combined market share of two suppliers), the following constraints must be met:

Σ_{iϵI}_{^{s}}Σ_{kϵK}_{^{s}}xₕᵢₖ+Σ_{iϵI}_{^{s′}}Σ_{kϵK}_{^{s′}}xₕᵢₖ−Σ_{sϵS}Σ_{iϵI}_{^{s}}Σ_{kϵK}_{^{s}}xₕᵢₖ·k(v)≥( [symbol] _{hss′k}−1)·M

∀hϵH,kϵK^{s},s,s′ϵS,s≠s′ (8)

eₕₛₖ≤Σ_{s′ϵS∩s′≠s} [symbol] _{hss′k}∀hϵH,kϵK^{s},sϵS (9)

where

[symbol] _{hss′k} is an interim binary variable; [symbol] _{hss′k} is 1 if the combined market share of suppliers s and s′ is equal to or higher than the minimum market share requirement, k(v), placed on tier k by supplier s, and 0 otherwise.

Similarly, when k(w)=3 (i.e., the combined market share of three suppliers), the following constraints must be met.

Σ_{iϵI}_{^{s}}Σ_{kϵK}_{^{s}}xₕᵢₖ+Σ_{iϵI}_{^{s′}}Σ_{kϵK}_{^{s′}}xₕᵢₖ+Σ_{iϵI}_{^{s″}}Σ_{kϵK}_{^{s″}}xₕᵢₖ−Σ_{sϵS}Σ_{iϵI}_{^{s}}Σ_{kϵK}_{^{s}}xₕᵢₖ·k(v)≥( [symbol] _{hss′s″k}−1)·M

∀hϵH,kϵK^{s},s,s′,s″ϵS,s≠s′≠s″ (10)

eₕₛₖ≤Σ_{(s′,s″)ϵS∩(s′≠s″≠s)} [symbol] _{hss′s″k}

∀hϵH,kϵK^{s},s,s′ϵS,s≠s′ (11)

where

[symbol] _{hss″s″k} is a binary variable; [symbol] _{hss″s″k} is 1 if the combined market share of three suppliers (s, s′, and s″) is equal to or higher than the minimum market share requirement placed on tier k by supplier s, and 0 otherwise.

Constraints (8) and (10) prevent a facility from choosing a tier of which the minimum combined market share requirement is not met.

Consider now the IDN-based discount schedule. To qualify for an IDN tier, the consolidated purchase of all hospitals must meet the minimum single market share requirement placed by supplier s, as described in the constraint below:

Σ_{hϵH}Σ_{iϵI}_{^{s}}Σ_{kϵK}_{^{s}}xₕᵢₖ−Σ_{hϵH}Σ_{sϵS}Σ_{iϵI}_{^{s}}Σ_{kϵK}_{^{s}}xₕᵢₖ·k(u)≥(qₛₖ−1)·M

∀hϵH,kϵK^{s},s,sϵS (12)

The combined market share requirement at the IDN level may also involve two or three suppliers including supplier s. When k(w)=2 (i.e., the combined market share of two suppliers), the following constraints must be met:

Σ_{hϵH}Σ_{iϵI}_{^{s}}Σ_{kϵK}_{^{s}}xₕᵢₖ+Σ_{hϵH}Σ_{iϵI}_{^{s′}}Σ_{kϵK}_{^{s′}}xₕᵢₖ−Σ_{hϵH}Σ_{sϵS}Σ_{iϵI}_{^{s}}Σ_{kϵK}_{^{s}}xₕᵢₖ·k(v)≥(θ_{ss′k}−1)·M ∀kϵK^{s},s,s′ϵS,s≠s′ (13)

qₛₖ≤Σ_{s′ϵS∩s′≠s}θ_{ss′k} ∀kϵK^{s},sϵS (14)

where

θ_{ss′k} is a binary variable; θ_{ss′k} is 1 if the combined market share of suppliers s and s′ across all hospitals in an IDN meets the minimum market share requirement placed on tier k by supplier s, and 0 otherwise.

qₛₖ is a binary variable; qₛₖ equals 1 if the IDN qualifies for tier k offered by supplier s, and 0 otherwise.

In the case where the combined market share is measured on three suppliers (i.e., k(w)=3), the following constraints are added:

Σ_{hϵH}Σ_{iϵI}_{^{s}}Σ_{kϵK}_{^{s}}xₕᵢₖ+Σ_{hϵH}Σ_{iϵI}_{^{s′}}Σ_{kϵK}_{^{s′}}xₕᵢₖ+Σ_{hϵH}Σ_{iϵI}_{^{s″}}Σ_{kϵK}_{^{s″}}xₕᵢₖ−Σ_{hϵH}Σ_{sϵS}Σ_{iϵI}_{^{s}}Σ_{kϵK}_{^{s}}xₕᵢₖ·k(v)≥(ζ_{hss′s″k}−1)·M

∀kϵK^{s},s,s′,s″ϵS,s≠s′≠s″ (15)

qₛₖ≤Σ_{(s′,s″)ϵS∩(s′≠s″≠s)}ζ_{hss′s″k} ∀kϵK^{s},s,s′ϵS,s≠s′ (16)

where

ζ_{ss′s″k} is a binary variable, describing whether the combined market share of three suppliers (s, s′, and s″) satisfy the minimum market share requirement placed on tier k by supplier s.

Constraints (13) and (15) guarantees that an IDN tier cannot be considered unless the minimum combined market share requirement is met.

Another complication in the medical supply tier contract is the mutually exclusive relationship between the IDN and the facility discount schedules. Constraint (2) and the following constraint combine to ensure that only one of the two discount schedules is selected.

Σ_{hϵH}eₕₛₖ≤|H|·qₛₖ ∀ϵK^{s},sϵS,k(z)=IDN (17)

The MILP model (1)-(16) addresses the product substitute decision, products and quantity to be purchased from each supplier, and the optimal tier prices that each facility shall adopt such that the total medical supply costs of the IDN are minimized. The MILP model solver 242 may apply an out-of-the-box solver, such as IBM ILOG CPLEX, Gurobi, FICOR Xpress, SCIP, etc., to find an optimal or near-optimal solution to the MILP model disclosed herein for a single stand-alone hospital, a regional IDN with dozens of facilities, or even a nationwide IDN with over a hundred of member hospitals.

In the case where certain physician preferred items cannot be substituted with alternatives, the physician preference restriction 261 is input to the hospital supply spend optimizer 240. The following constraint is an example to ensure that the facility purchases physician preferred items.

Σ_{kϵK}_{^{s}}xₕᵢₖ≥θₕᵢ ∀hϵH,sϵS,iϵI^{s} (18)

where

θₕᵢ is the forecast annual demand of physician preferred item i at facility h, ∀hϵH, iϵI.

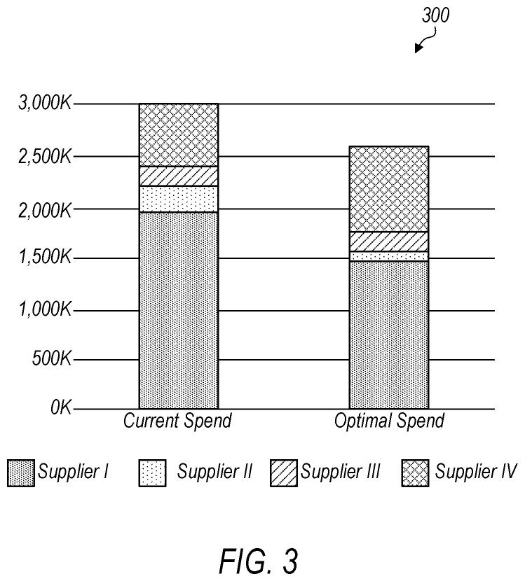

Turning to FIG. 3, it shows an example of change in spends on a healthcare clinical product category after applying the hospital supply spend optimization system 200 to a regional IDN that purchases from four contract vendors. This IDN has the opportunity to achieve 14% cost savings without physician preference restrictions.

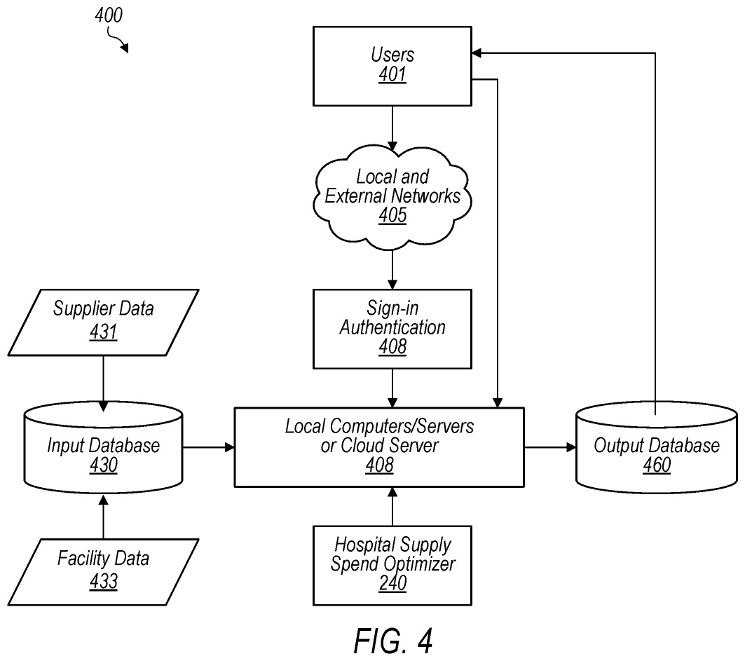

FIG. 4 illustrates an example computer-implemented environment 400 for implementing the disclosed embodiments. The hospital supply spend optimizer 240 may be stored on local computers or on cloud server 420. Users may run the optimizer 240 either on the local computer, or on the local server through local network 405 by providing sign-in credentials 408, or on cloud server through internet by providing sign-in credentials 408. Data from suppliers 431 and facility data (e.g., data from member hospitals) 433 are stored in one or more computers, database, or input database 430. The local computer, local server, or cloud server has access to the input database 430, and feed the supplier data 431 and facility data 433 to the hospital supply spend optimizer 240. The results generated by the optimizer 240, including the spend data, product substitute decision, products and quantity to be purchased from each supplier, and the tier selection decision, are stored in one or more computers, database, or storage media 460, and are accessible to users 401 for review.

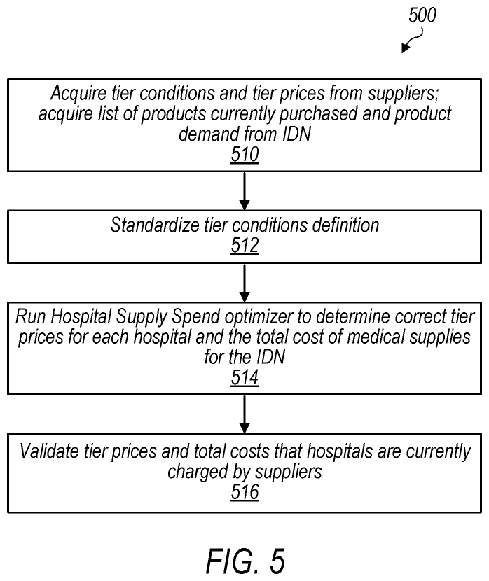

FIG. 5 describes a method 500 with example steps of using the disclosed embodiments to validate prices charged by suppliers to member hospitals in an IDN. Information regarding tier conditions and product tier prices offered by suppliers, and product demand of member hospitals, is acquired at step 510. Tier requirements from all suppliers are standardized to multi-dimensional vectors at step 512. The hospital supply spend optimizer calculates the tier prices for which hospitals qualify and selects the tiers that give the IDN the lowest total medical supply cost at step 514. Finally, at step 516, tier prices obtained at step 514 are compared with the prices that hospitals are currently paying; any discrepancies are reviewed by IDN or facility operation team for correction.

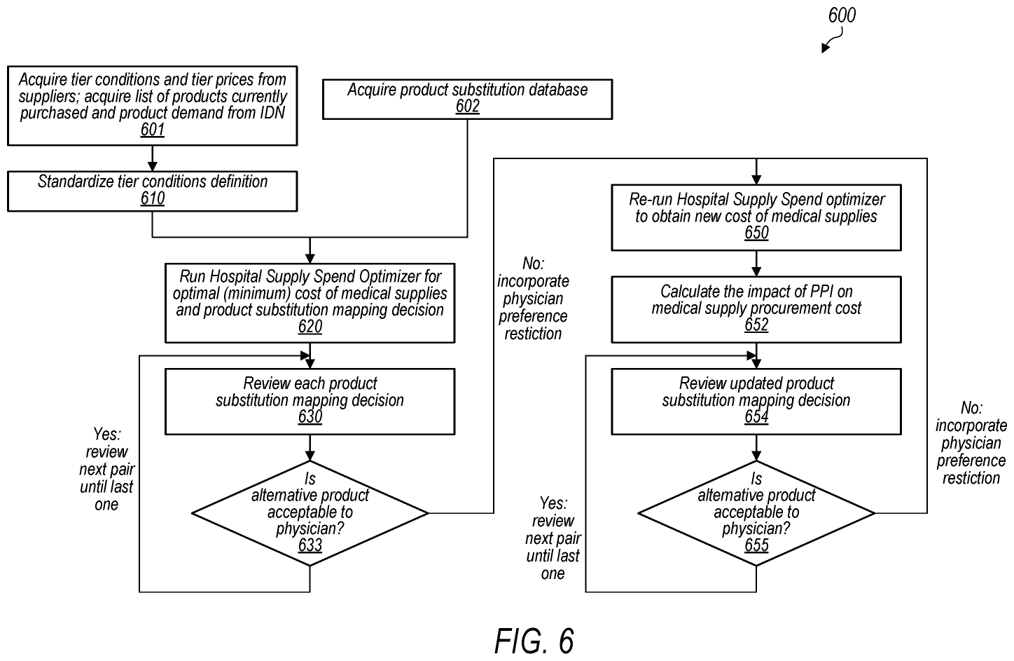

An example process 600 for evaluating the impact of physician preference items on hospital procurement costs is presented in FIG. 6. It starts by acquiring tier definitions and product tier prices offered by suppliers, the forecast product demand of member hospitals, and a product substitution database, as described at steps 601 and 602. Tier requirements from all suppliers are standardized to multi-dimensional vectors at step 610. The hospital supply spend optimizer is executed at step 620 for product substitution mapping decision and obtaining the minimum possible cost of medical supplies. The model-recommended cost-minimization product substitution mapping is reviewed to determine whether the recommended alternative products are acceptable to physicians, as shown at steps 630 and 633. The pairs—the products currently in use and the recommended replacements—are reviewed one by one. If a pair is acceptable, the process repeats from step 630 until all the pairs are reviewed. If a pair is not acceptable to a physician, a physician preference restriction is incorporated into the hospital supply spend optimizer, and new medical supply cost and production substitution mapping are generated by the optimizer at step 650. By comparing the total cost generated from step 620 to the cost from step 650, the impact of the physician preference item is quantified in terms of medical supply procurement cost in 652. This sets a benchmark for physicians and hospitals' management team to assess whether standardizing a product to a substitute product is financially justified.

The updated product substitution mapping is again reviewed to determine whether the recommended alternative products are acceptable to physicians, as described at steps 654 and 655. If a pair, including the product currently in use and the recommended replacement, is acceptable, the process repeats from step 654 unless all the pairs have been reviewed. If a pair is not acceptable, a new physician preference restriction is incorporated into the hospital supply spend optimizer and the process repeats from step 650.

### Example Methods

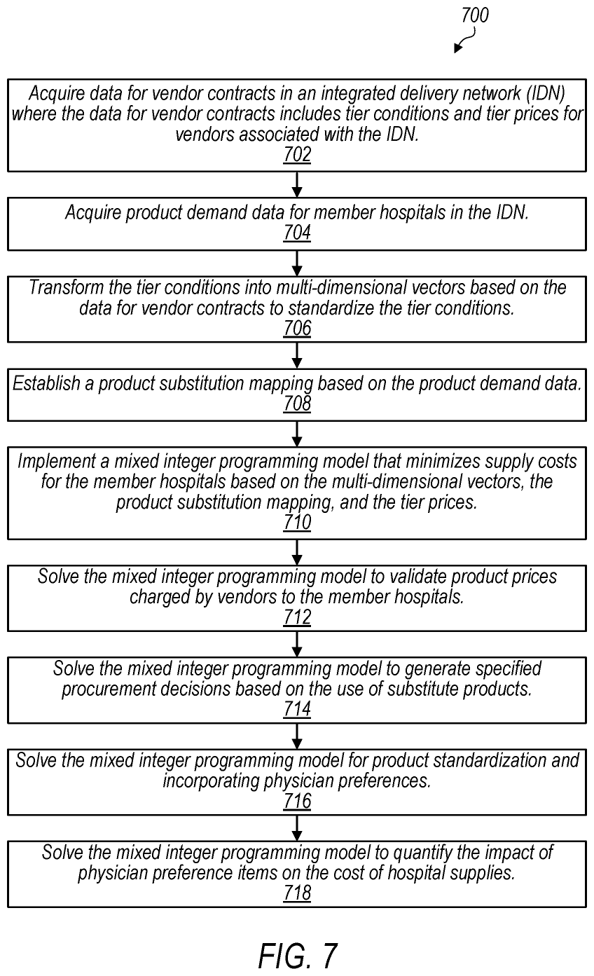

FIG. 7 is a flow diagram illustrating a method for reducing hospital supply costs, according to some embodiments. Method 700 shown in FIG. 7 may be used in conjunction with any of the computer circuitry, systems, devices, elements, or components disclosed herein, among other devices. In various embodiments, some of the method elements shown may be performed concurrently, in a different order than shown, or may be omitted. Additional method elements may also be performed as desired. In various embodiments, some or all elements of this method may be performed by a particular computer system, such as computing device 810, described below.

At 702, in the illustrated embodiment, a computer system acquires data for vendor contracts in an integrated delivery network (IDN) where the data for vendor contracts includes tier conditions and tier prices for vendors associated with the IDN.

At 704, in the illustrated embodiment, the computer system acquires product demand data for member hospitals in the IDN.

At 706, in the illustrated embodiment, the computer system transforms the tier conditions into multi-dimensional vectors based on the data for vendor contracts to standardize the tier conditions.

At 708, in the illustrated embodiment, the computer system establishes a product substitution mapping based on the product demand data.

At 710, in the illustrated embodiment, the computer system implements a mixed integer programming model that minimizes supply costs for the member hospitals based on the multi-dimensional vectors, the product substitution mapping, and the tier prices.

At 712, in the illustrated embodiment, the computer system solves the mixed integer programming model to validate product prices charged by vendors to the member hospitals.

At 714, in the illustrated embodiment, the computer system solves the mixed integer programming model to generate specified procurement decisions based on the use of substitute products.

At 716, in the illustrated embodiment, the computer system solves the mixed integer programming model for product standardization and incorporating physician preferences.

At 718, in the illustrated embodiment, the computer system solves the mixed integer programming model to quantify the impact of physician preference items on the cost of hospital supplies.

### Example Computer System

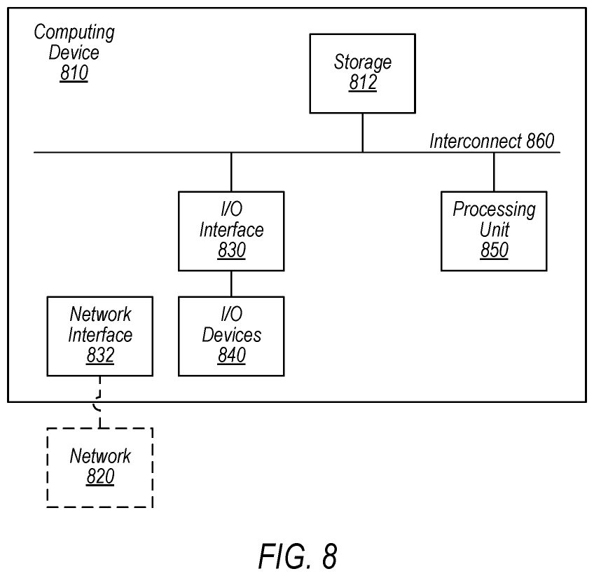

Turning now to FIG. 8, a block diagram of one embodiment of computing device (which may also be referred to as a computing system) 810 is depicted. Computing device 810 may be used to implement various portions of this disclosure. Computing device 810 may be any suitable type of device, including, but not limited to, a personal computer system, desktop computer, laptop or notebook computer, mainframe computer system, web server, workstation, or network computer. As shown, computing device 810 includes processing unit 850, storage 812, and input/output (I/O) interface 830 coupled via an interconnect 860 (e.g., a system bus). I/O interface 830 may be coupled to one or more I/O devices 840. Computing device 810 further includes network interface 832, which may be coupled to network 820 for communications with, for example, other computing devices.

In various embodiments, processing unit 850 includes one or more processors. In some embodiments, processing unit 850 includes one or more coprocessor units. In some embodiments, multiple instances of processing unit 850 may be coupled to interconnect 860. Processing unit 850 (or each processor within 850) may contain a cache or other form of on-board memory. In some embodiments, processing unit 850 may be implemented as a general-purpose processing unit, and in other embodiments it may be implemented as a special purpose processing unit (e.g., an ASIC). In general, computing device 810 is not limited to any particular type of processing unit or processor subsystem.

As used herein, the term “module” refers to circuitry configured to perform specified operations or to physical non-transitory computer readable media that store information (e.g., program instructions) that instructs other circuitry (e.g., a processor) to perform specified operations. Modules may be implemented in multiple ways, including as a hardwired circuit or as a memory having program instructions stored therein that are executable by one or more processors to perform the operations. A hardware circuit may include, for example, custom very-large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, or the like. A module may also be any suitable form of non-transitory computer readable media storing program instructions executable to perform specified operations.

Storage 812 is usable by processing unit 850 (e.g., to store instructions executable by and data used by processing unit 850). Storage 812 may be implemented by any suitable type of physical memory media, including hard disk storage, floppy disk storage, removable disk storage, flash memory, random access memory (RAM-SRAM, EDO RAM, SDRAM, DDR SDRAM, RDRAM, etc.), ROM (PROM, EEPROM, etc.), and so on. Storage 812 may consist solely of volatile memory, in one embodiment. Storage 812 may store program instructions executable by computing device 810 using processing unit 850, including program instructions executable to cause computing device 810 to implement the various techniques disclosed herein.

I/O interface 830 may represent one or more interfaces and may be any of various types of interfaces configured to couple to and communicate with other devices, according to various embodiments. In one embodiment, I/O interface 830 is a bridge chip from a front-side to one or more back-side buses. I/O interface 830 may be coupled to one or more I/O devices 840 via one or more corresponding buses or other interfaces. Examples of I/O devices include storage devices (hard disk, optical drive, removable flash drive, storage array, SAN, or an associated controller), network interface devices, user interface devices or other devices (e.g., graphics, sound, etc.).

Various articles of manufacture that store instructions (and, optionally, data) executable by a computing system to implement techniques disclosed herein are also contemplated. The computing system may execute the instructions using one or more processing elements. The articles of manufacture include non-transitory computer-readable memory media. The contemplated non-transitory computer-readable memory media include portions of a memory subsystem of a computing device as well as storage media or memory media such as magnetic media (e.g., disk) or optical media (e.g., CD, DVD, and related technologies, etc.). The non-transitory computer-readable media may be either volatile or nonvolatile memory.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

What is claimed is:

1. A method for reducing hospital supply costs, comprising:
acquiring, by a computer processor, data for vendor contracts in an integrated delivery network (IDN), wherein the data for vendor contracts includes tier conditions and tier prices for vendors associated with the IDN;
acquiring, by the computer processor, product demand data for member hospitals in the IDN;
transforming, by the computer processor, the tier conditions into multi-dimensional vectors based on the data for vendor contracts to standardize the tier conditions;
establishing, by the computer processor, a product substitution mapping based on the product demand data;
implementing, by the computer processor, a mixed integer programming model that minimizes hospital supply costs for the member hospitals based on the multi-dimensional vectors, the product substitution mapping, and the tier prices;
solving, by the computer processor, the mixed integer programming model to validate product prices charged by the vendors to the member hospitals;
solving, by the computer processor, the mixed integer programming model to generate specified procurement decisions based on uses of substitute products according to the product substitution mapping;
solving, by the computer processor, the mixed integer programming model for product standardization and incorporating physician preferences; and
solving, by the computer processor, the mixed integer programming model to quantify impacts of physician preference items on the hospital supply costs.

2. The method of claim 1, wherein the vendor contracts are proposed by at least one of a healthcare group purchasing organization (GPO) and suppliers, and wherein the vendor contracts have been agreed to by a healthcare provider in the IDN.

3. The method of claim 1, wherein the tier conditions are transformed into a six-dimensional vector.

4. The method of claim 1, wherein transforming the tier conditions into the multi-dimensional vectors standardizes the tier conditions across all healthcare clinical product categories in the IDN.

5. The method of claim 1, wherein the product substitution mapping is a one-way substitution mapping, determined based on input from suppliers or the member hospitals in the IDN.

6. The method of claim 1, wherein results from solving the mixed integer programming model by permitting the uses of substitute products include: minimized hospital supply costs for the IDN and cost distribution among the member hospitals, optimal spends on individual vendors, product and quantity to be purchased from each vendor, tier prices qualified and assigned to member hospitals, and recommended product substitution structure for cost minimization.

7. The method of claim 1, wherein results from solving the mixed integer programming model without taking into account the product substitution mapping is used to determine or validate prices paid by the member hospitals.

8. The method of claim 1, further comprising adding, one at a time, physician preference constraints to the mixed integer programming model, wherein the model is solved to quantify an impact of a physician preferred item on the hospital supply costs.

9. A system for reducing hospital supply costs, the system comprising:
an input database that stores a plurality of vendor contracts and hospitals' product demand data associated with an integrated delivery network (IDN);
an analysis unit comprising a computer processor and memory, wherein the analysis unit is operable to:
acquire data for the vendor contracts from the input database, wherein the data for the vendor contracts includes tier conditions and tier prices for vendors associated with the IDN;
acquire product demand data for member hospitals in the IDN;
transform the tier conditions into multi-dimensional vectors based on the data for vendor contracts to standardize the tier conditions;
establish a product substitution mapping based on the product demand data;
implement a mixed integer programming model that minimizes hospital supply costs for the member hospitals based on the multi-dimensional vectors, the product substitution mapping, and the tier prices;
solve the mixed integer programming model to validate product prices charged by vendors to the member hospitals;
solve the mixed integer programming model to generate specified procurement decisions based on uses of substitute products;
solve the mixed integer programming model for product standardization and incorporating physician preferences; and
solve the mixed integer programming model to quantify impacts of physician preference items on the hospital supply costs.

10. The system of claim 9, wherein the vendor contracts are proposed by at least one of a healthcare group purchasing organization (GPO) and suppliers, and wherein the vendor contracts have been agreed to by a healthcare provider in the IDN.

11. The system of claim 9, wherein the analysis unit is configured to transform the tier conditions into a six-dimensional vector.

12. The system of claim 9, wherein the analysis unit is configured to transform the tier conditions into the multi-dimensional vectors standardizes the tier conditions across all healthcare clinical product categories in the IDN.

13. The system of claim 9, wherein the analysis unit is operable to establish one-way product substitution mapping based on input from suppliers or the member hospitals in the IDN.

14. The system of claim 9, further comprising an output dataset that stores results from solving the mixed integer programming model by permitting the uses of substitute products, including: minimized hospital supply costs for the IDN and cost distribution among member hospitals, optimal spends on individual vendors, product and quantity to be purchased from each vendor, tier prices qualified and assigned to member hospitals, and recommended product substitution structure for cost minimization.

15. The system of claim 9, wherein the analysis unit is configured to use results from solving the mixed integer programming model without taking into account product substitution mapping to determine or validate prices paid by the member hospitals.

16. The system of claim 9, wherein the analysis unit is configured to incorporate physician preference constraints, one at a time, to the mixed integer programming model, and then, solve an model to quantify the impact of a physician preferred item on the hospital supply costs.

17. A non-transitory computer-readable medium having instructions stored thereon that are executable by a computing device to perform operations, comprising:
acquiring data for vendor contracts in an integrated delivery network (IDN), wherein the data for vendor contracts includes tier conditions and tier prices for vendors associated with the IDN;
acquiring product demand data for member hospitals in the IDN;
transforming the tier conditions into multi-dimensional vectors based on the data for vendor contracts to standardize the tier conditions;
establishing a product substitution mapping based on the product demand data;
implementing a mixed integer programming model that minimizes hospital supply costs for the member hospitals based on the multi-dimensional vectors, the product substitution mapping, and the tier prices;
solving the mixed integer programming model to validate product prices charged by the vendors to the member hospitals;
solving the mixed integer programming model to generate specified procurement decisions based on uses of substitute products according to the product substitution mapping;
solving the mixed integer programming model for product standardization and incorporating physician preferences; and
solving the mixed integer programming model to quantify impacts of physician preference items on the hospital supply costs.

18. The non-transitory computer-readable medium of claim 17, wherein the vendor contracts are proposed by at least one of a healthcare group purchasing organization (GPO) and suppliers, and wherein the vendor contracts have been agreed to by a healthcare provider in the IDN.

19. The non-transitory computer-readable medium of claim 17, wherein transforming the tier conditions into the multi-dimensional vectors standardizes the tier conditions across all healthcare clinical product categories in the IDN.

20. The non-transitory computer-readable medium of claim 17, wherein the product substitution mapping is a one-way substitution mapping, determined based on input from suppliers or the member hospitals in the IDN.
